(19) United States
(12) Patent Application Publication
(10) Pub. No.: US 2025/0120489 A1
(43) Pub. Date: Apr. 17, 2025

(54) CLEANING AND DISINFECTING DEVICE

(71) Applicants: RICARDO A. IGLESIAS, DADE CITY, FL (US); ANTHONY A. IGLESIAS, DADE CITY, FL (US)

(72) Inventors: RICARDO A. IGLESIAS, DADE CITY, FL (US); ANTHONY A. IGLESIAS, DADE CITY, FL (US)

(21) Appl. No.: 18/907,553

(22) Filed: Oct. 6, 2024

Related U.S. Application Data
(60) Provisional application No. 63/544,320, filed on Oct. 16, 2023.

(51) Int. Cl. A45D 27/46; A61L 2/18; A61L 2/26; A61L 101/32; B08B 3/08; B08B 3/14

(52) U.S. Cl. CPC A45D 27/46; A61L 2/18; A61L 2/26; B08B 3/08; B08B 3/14; A61L 2101/32; A61L 2202/121; A61L 2202/122; A61L 2202/15; A61L 2202/17

(57) ABSTRACT

A cleaning and disinfecting device comprises a tank having an open top end providing access into a first interior space configured to receive therein a fluid, such as a cleaning and disinfecting/sterilizing fluid. An instrument support structure is configured to be removably inserted into the first interior space of the tank for supporting at least one instrument to be cleaned and disinfected/sterilized by the fluid. A compartment having a second interior space is disposed within the first interior space of the tank. Filter means is removably disposed in the second interior space of the compartment. The device further comprises means for circulating the fluid within the first interior space of the tank through the filter means to filter the fluid by removing therefrom debris collected by the fluid when cleaning and disinfecting the instrument.

### CROSS-REFERENCE TO RELATED APPLICATION

This application claims priority to U.S. Provisional Patent Application No. 63/544,320, filed Oct. 16, 2023.

### BACKGROUND

### Technical Field

The present invention relates generally to cleaning and disinfecting devices. More particularly, the present invention is directed to cleaning and disinfecting devices for cleaning and disinfecting various instruments used in the beauty industry, such as razor, shears, scissors, combs, brushes and the like used in the barber industry, as well as nail care instruments.

### Background Information

Under the law of most states, professionals in the beauty industry, such as barber and nail care professionals, are required to disinfect all instruments between their use on each customer. Most conventional disinfecting devices used for this purpose are simply containers (e.g., tanks or jars) containing a disinfecting fluid into which barber instruments (e.g., shears, combs, brushes, etc.) and nail care instruments are submerged for disinfection. This necessitates the removal of the instruments out of the fluid and wiping them before each use, including to remove all foreign substances, such as loose hair and the like for barber instruments, which ordinarily accumulates in most disinfecting fluids. This is not only unsanitary but also consumes a great amount of time.

In the conventional disinfecting devices of the type described above, the disinfecting fluid must be changed daily in all containers used to submerge the instruments. In fact, it is recommended that the disinfecting fluid be changed out 24 hours after its first use. This is a further disadvantage associated with conventional disinfecting devices in terms of increased costs and time associated with the replacement of the disinfecting fluid.

Thus, it is desirable to provide a cleaning and disinfecting device which overcomes the foregoing drawbacks and problems associated with the conventional art.

### SUMMARY

In order to overcome the foregoing deficiencies in the conventional art, it is a primary object of the present invention to disclose a cleaning and disinfecting device especially adapted for use with, but not limited to, barber and nail care instruments, in which the instruments may be placed and continuously and automatically cleaned and disinfected in a minimum amount of time and at a lower cost as compared to the conventional art.

In order to carry out the preceding and other objects, a cleaning and disinfecting device according to an embodiment of the present invention comprises a tank having an open top end providing access into a first interior space configured to receive therein a fluid, such as a cleaning and disinfecting/sterilizing fluid. An instrument support structure is configured to be removably inserted into the first interior space of the tank for supporting at least one instrument to be cleaned and disinfected/sterilized by the fluid. A compartment having a second interior space is disposed within the first interior space of the tank. Filter means is removably disposed in the second interior space of the compartment. The device further comprises means for circulating the fluid within the first interior space of the tank through the filter means to filter the fluid by removing therefrom debris collected by the fluid when cleaning and disinfecting the instrument.

According to features of the present invention, the device further comprises means for removably inserting and lifting the instrument support means into and out of the first interior space of the tank. The instrument support means comprises a rack having at least one compartment for supporting the instrument. The means for removably inserting and lifting the instrument support means comprises a cover integrally connected to the rack for covering the open top end of the tank.

According to other features of the present invention, the means for circulating the fluid comprises a flow pipe having an inlet opening, a pipe fitting assembly having an outlet opening in fluid communication with the flow pipe, and a pump assembly for pumping the fluid disposed in the first interior space of the tank through the flow pipe via the inlet opening and out of the outlet opening of the pipe fitting assembly. The filter means comprises a filter element that separates the second interior space into a first compartment section and a second compartment section. The pipe fitting assembly is disposed in the first compartment section and the pump assembly has an intake and discharge port opening into the first compartment section. The pumped fluid flows into the first compartment section and through the filter means to filter the fluid.

As yet another feature of the present invention, the compartment is disposed proximate the open top end of the tank and has a wall provided with an undercut portion defining an overflow region of the compartment via which the filtered fluid is allowed to flow in a controlled manner downwardly back into the first interior space of the tank.

According to an exemplary embodiment, the fluid comprises a cleaning and disinfecting fluid, such as Barbicide®. The fluid may also be any other type of commercially available disinfectant or sterilizer.

In other exemplary embodiments, the instrument comprises instruments used in the beauty industry, such as barber instruments and/or nail care instruments.

As yet another exemplary embodiment, each of the tank and the compartment comprises walls made of a transparent material.

The foregoing and other features and advantages of the cleaning and disinfecting device according to embodiments the present invention will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a perspective view of a cleaner and disinfecting device according to a first embodiment of the present invention.

FIG. 2 is front view of the device in FIG. 1.

FIG. 3 is rear view of the device in FIG. 1.

FIG. 4 is a left side view of the device in FIG. 1.

FIG. 5 is a top view of the device in FIG. 1.

FIG. 6 is bottom view of the device in FIG. 1.

FIG. 7 is an exploded view of the device in FIG. 1.

FIG. 8 is a front view of an embodiment of instrument support means and cover assembly for the device in FIG. 1.

FIG. 9 is a front view illustrating an assembly of structural components of the device in FIG. 1, including a filter compartment and elements defining means for circulating a disinfecting fluid through a filter means provided in the filter compartment.

FIG. 10 is a side view illustrating a positional relationship between the assembly in FIG. 9 and the instrument support means and cover assembly of the device in FIG. 1.

FIG. 11 is a perspective view of the container of the device in FIG. 1, with front and left-side walls of the container being omitted to illustrate the interior of the container and an opening formed in a rear wall of the container for mounting components of the means for circulating the disinfecting fluid.

FIG. 12 is an illustration showing a perspective view of the assembly in FIG. 9, with front and left-side walls of the filter compartment being removed to illustrate the filter means disposed in the filter compartment.

FIG. 13 is a front view of the illustration in FIG. 12.

FIG. 14 is a view similar to FIG. 10, with a left-side wall of the filter compartment being removed to illustrate a positional relationship between the filter means and the means for circulating the disinfecting fluid through the filter means.

FIG. 15 is an illustration showing a perspective view of the assembly shown in FIGS. 9, 10 and 13 being mounted to the container shown in FIG. 11, with the instrument support means and cover assembly being removed for a better understanding of the illustration.

FIG. 16 is a front view of the illustration in FIG. 15.

FIG. 17 is a rear view of the illustration in FIG. 15.

FIG. 18 is a left-side view of the illustration in FIG. 15.

FIG. 19 is a top view of the illustration in FIG. 15.

FIG. 20 is a bottom view of the illustration in FIG. 15.

FIG. 21 is an exploded view of a cleaner and disinfecting device according to a second embodiment of the present invention.

FIG. 22 is a front view of an embodiment of instrument support means and cover assembly for the device in FIG. 21.

FIG. 23 is a front view illustrating an assembly of structural components of the device in FIG. 21, including a filter compartment and elements defining means for circulating a disinfecting fluid through a filter means provided in the filter compartment.

FIG. 24 is a side view illustrating a positional relationship between the assembly in FIG. 23 and the instrument support means and cover assembly of the device in FIG. 21.

FIG. 25 is a perspective view of the device in FIG. 21 shown in an assembled state and with the instrument support means and cover assembly being removed for illustration purposes only.

FIG. 26 is a front view of the device in FIG. 25;

FIG. 27 is a rear view of the device in FIG. 25;

FIG. 28 is a left side view of the device in FIG. 25;

FIG. 29 is a top view of the device in FIG. 25;

FIG. 30 is a bottom view of the device in FIG. 25;

FIG. 31a is a perspective view of a pipe fitting assembly defining a component of the means for circulating a disinfecting fluid through a filter means for the cleaning and disinfecting device according to the first and second embodiments of the present invention.

FIG. 31b is a front view of the pipe fitting assembly in FIG. 31a.

FIG. 32 is a side view illustrating a positional relationship between the pipe fitting assembly of FIGS. 31a, 31b and a pump assembly for the cleaning and disinfecting device according to the first and second embodiments of the present invention.

### DETAILED DESCRIPTION OF PREFERRED EMBODIMENTS

It is to be understood that the foregoing general description and the following detailed description are exemplary and explanatory only and are not restrictive of the invention as claimed. The accompanying drawings, which are incorporated in and constitute a part of the specification, illustrate embodiments of the invention and together with the general description, serve to explain the principles of the invention. Throughout the drawings, the same reference numerals denote the same elements.

The present description may use perspective-based language such as front/rear, right/left, and top/bottom. Such language and language of similar import is merely used to facilitate the discussion, showing relative orientation and location of components to one another, and is not intended to restrict the application of embodiments of the present invention to particular orientations.

Embodiments of the present invention provide cleaning and disinfecting devices for cleaning and disinfecting various instruments used in the beauty industry, such as razors, shears, scissors, combs, brushes and the like used in the barber industry. The present invention is also adapted for cleaning and disinfecting instruments other than barber instruments, such as nail care instruments.

While the present invention is described with reference to “disinfecting” devices and corresponding use of “disinfectants,” the present invention is also intended to include “sterilizing” devices and corresponding “sterilizers.” In this regard, the present invention is intended to encompass both disinfection and sterilization as both relating to decontamination processes, with disinfection involving the process of eliminating or reducing harmful microorganisms from inanimate objects and surface, and sterilization involving the process of killing all microorganisms.

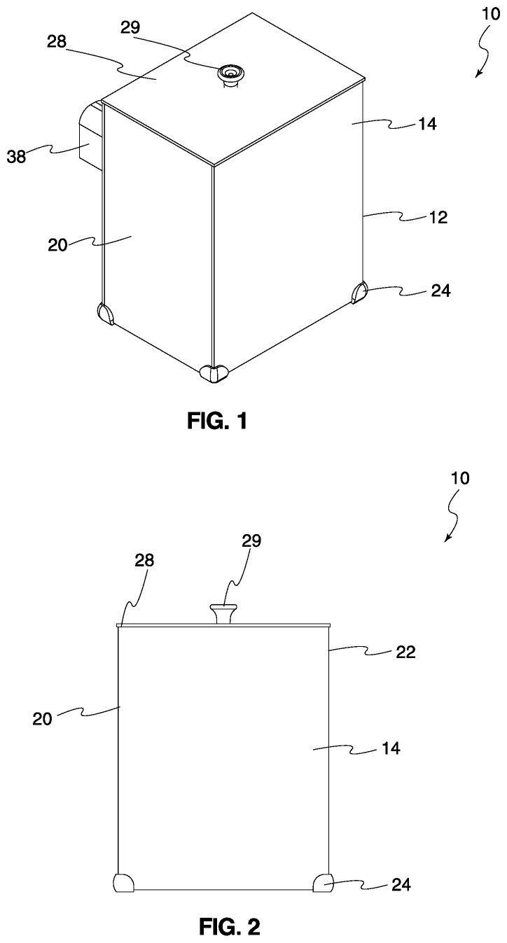

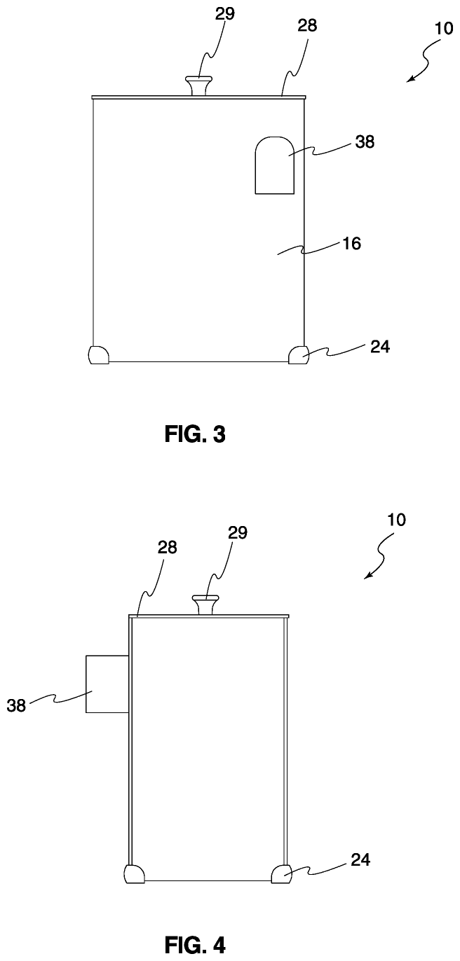

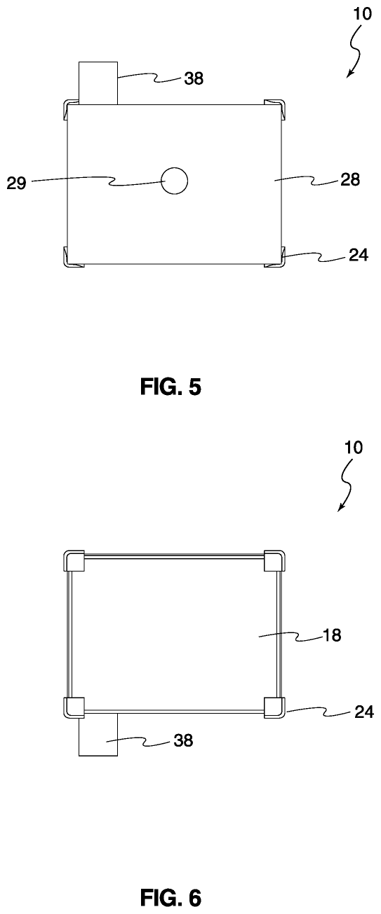

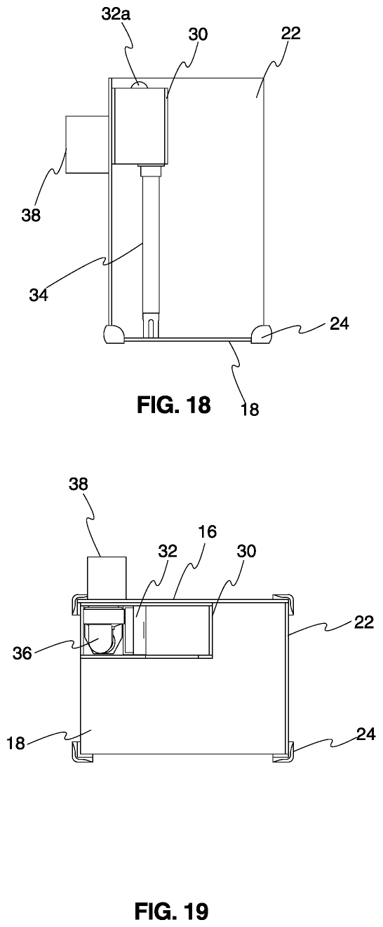

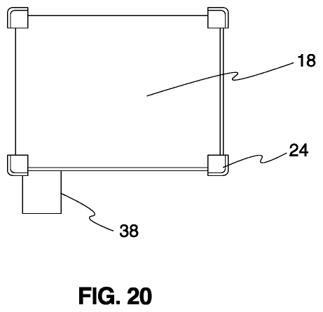

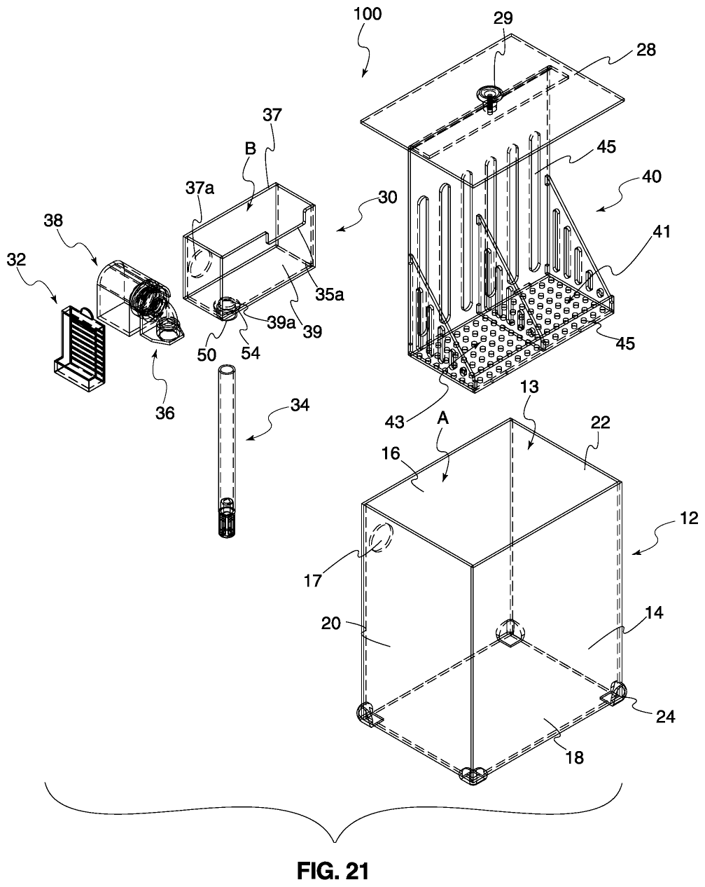

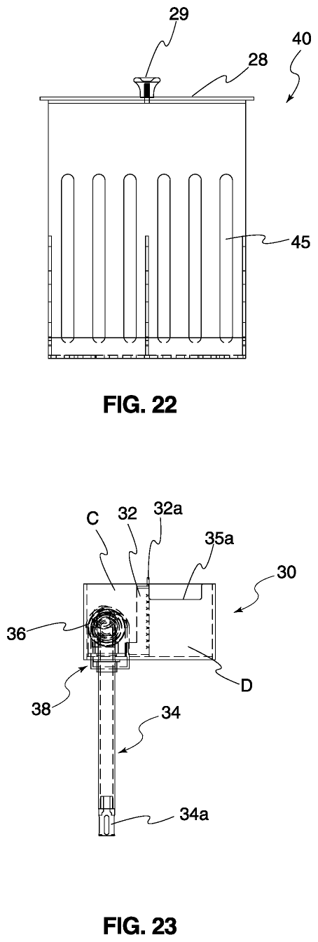

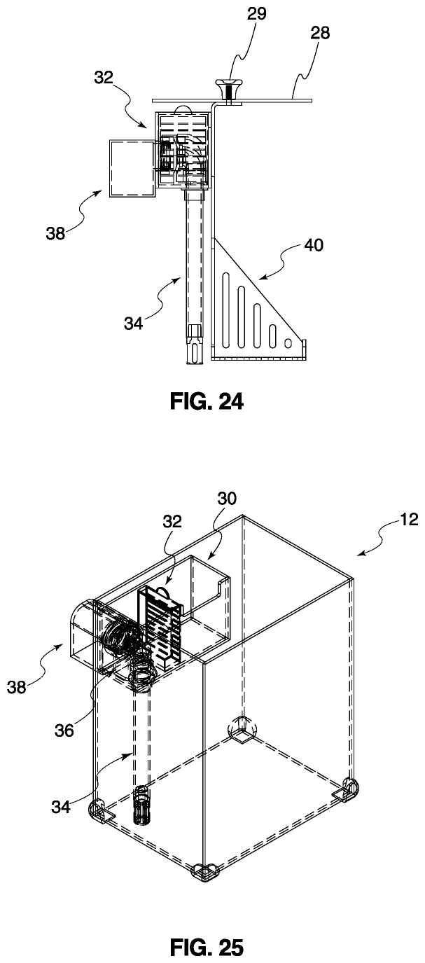

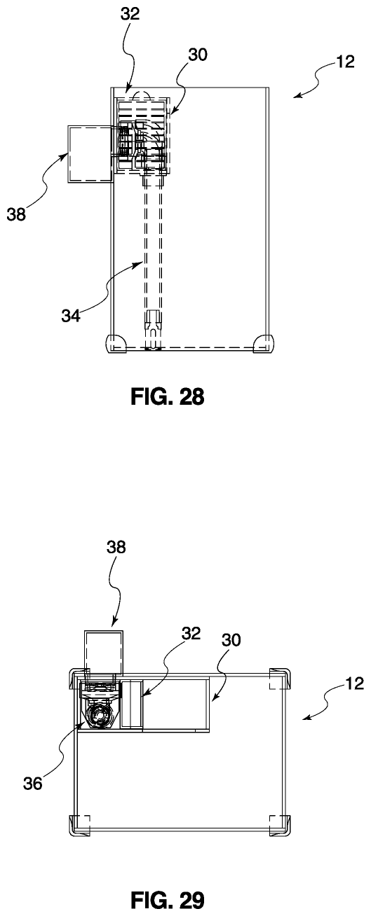

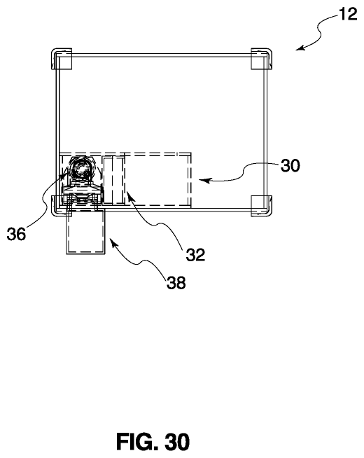

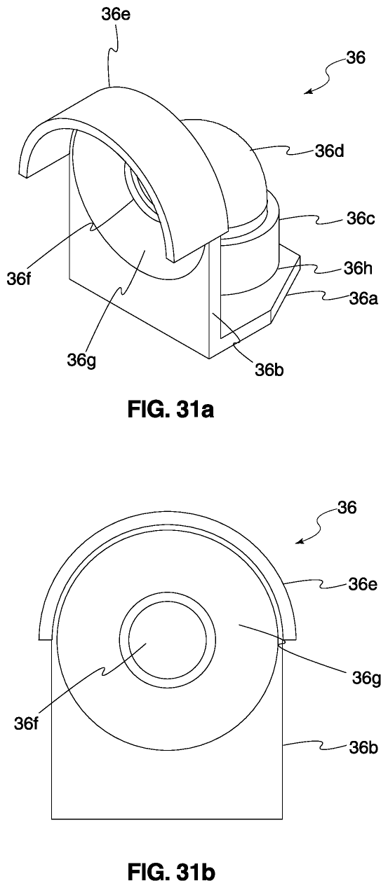

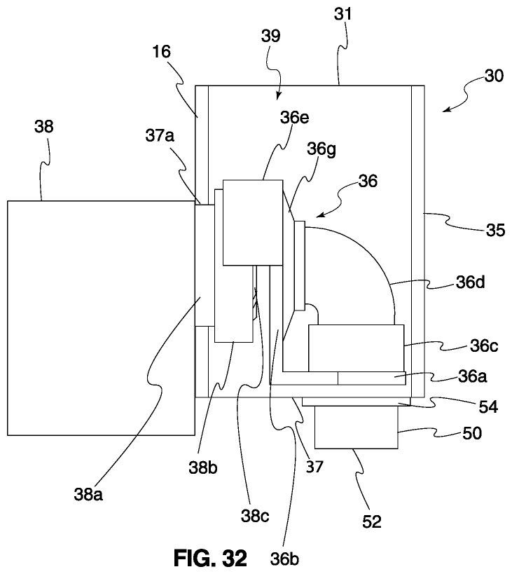

With reference now to the drawings, FIGS. 1-20 show an embodiment of the cleaning and disinfecting device, generally designated with numeral 10, according to a first embodiment of the present invention (hereinafter also “device 10”), FIGS. 21-30 show a second embodiment of the cleaning and disinfecting device, generally designated at 100 (hereinafter also “device 100”). In FIGS. 1-32, the same characters are used to designate the same parts. FIGS. 31a, 31b and 32 show details of components of means for circulating a cleaning and disinfecting fluid according to the first and second embodiments. The only difference between the first and second embodiments is that in the second embodiment a tank 12, cover 28, compartment 30 and instrument support means 40 of device 100 are made of a transparent material as compared to the corresponding components for device 10.

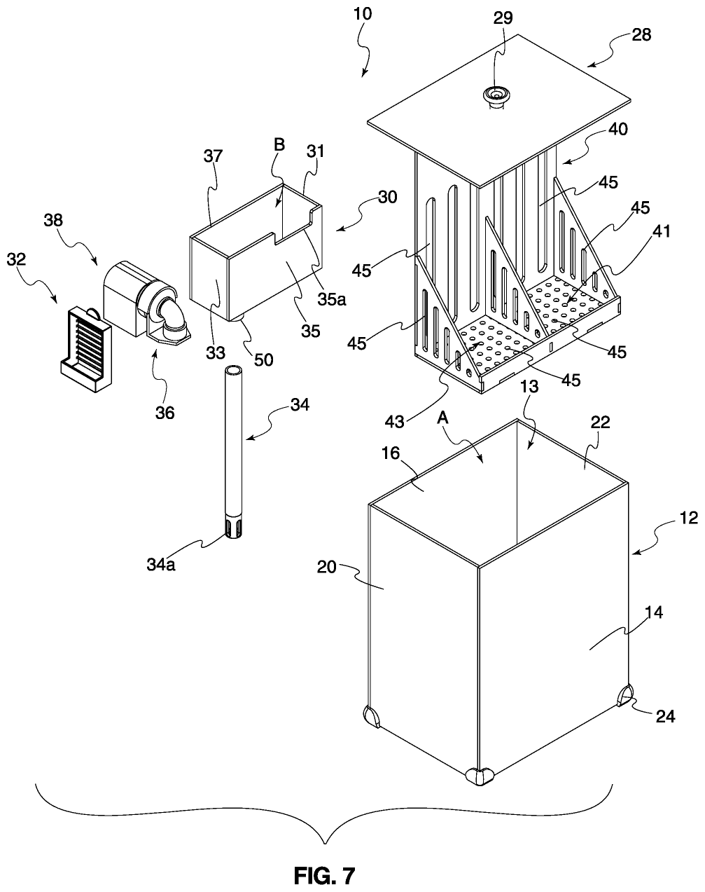

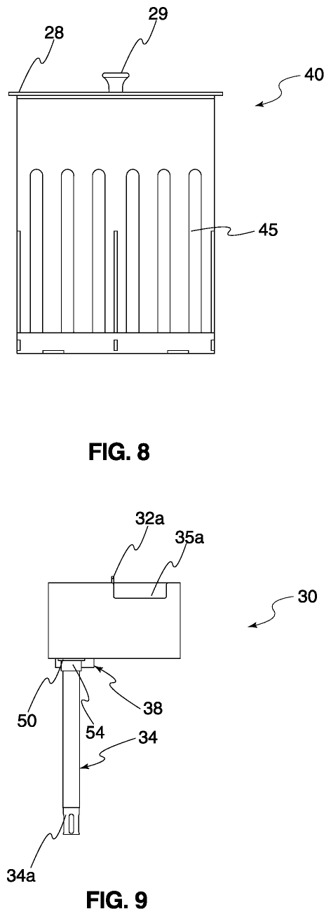

Referring to FIGS. 1-6, in this embodiment device 10 includes a container 12 forming a tank having a front wall 14, a rear wall 16, a bottom wall 18 defining a closed end of tank 12, and side walls 20, 22. Lower portions of walls 14, 16, 18 and 20 are optionally reinforced with reinforcement elements 24 as shown. Opposite to bottom wall 18, tank 12 has an open top end 13 (FIG. 7) configured to be covered by a removable cover 28 provided with a holding member 29 to facilitate manipulation of cover 28 during the process of covering and uncovering open top end 13.

FIG. 7 is an exploded view illustrating the components of device 10. Device 10 includes tank 12 as described above provided with an interior space A that is accessible through open top end 13. Interior space A of tank 12 is configured to receive via open top end 13 a fluid for cleaning and disinfecting various barber instruments, such as a disinfecting fluid as further described below. Device 10 is a compact structure including several components configured to be supported within and/or extend into interior space A of tank 12, including a compartment 30, a filter means 32, fluid flow pipe 34 having an inlet 34a, a pipe fitting assembly 36, a pump assembly 38, and instrument support means 40. Filter means 32 and assembly 36 are configured for placement inside an interior space B of compartment 30, adding to the compact structure of device 10. As further described below, flow pipe 34, fitting assembly 36 and pump assembly 38 define means for circulating a disinfecting fluid contained inside interior space A of tank 12 to clean the fluid by passing it through filter means 32 disposed in interior space B of compartment 30. The fluid is cleaned by action of filter means 32 which removes filtered particles from the fluid collected during cleaning and disinfection of instruments and related items as described below.

Referring to FIGS. 7 and 8, instrument support means 40 is configured to be removably inserted into interior space A of tank 12 for supporting various types of barber and/or nail care instruments. More specifically, instrument support means 40 include means 41, 43 for supporting or holding barber instruments including, but not limited to, scissors, razors, clippers, combs, tweezers as well as other related items. In the present embodiment, instrument support means 40 is in the form of a removable rack with means 41, 42 defining compartments for holding one or more of the various items described above. The configuration of rack 40 allows for complete immersion and cleaning and disinfection of the instruments by the disinfecting fluid contained in tank 12.

Rack 40 is provided with various openings denoted at 45 through which the fluid in interior space A of tank 12 is allowed to pass during insertion and removal of rack 40 into and from interior space A of tank 12. Stated otherwise, the flow of the fluid through openings 45 reduces the resistance applied by the fluid when rack 40 is moved or displaced within and relative to interior space A of tank 12, thereby facilitating the process of inserting and removing rack 40 into and from interior space A of tank 12.

Cover 28 with holding member 29 is securely mounted to a top end portion of rack 40. By this construction, it is appreciated that in addition to functioning as a cover for closing open top end 13 of tank 12, cover 28 provided with holding member 29 further define means for securely inserting and removing (e.g., lifting) rack 40 into and from interior space A of tank 12 containing the disinfecting fluid.

FIG. 9 is a front view of an assembly illustrating a positional relationship between fluid flow pipe 34 and pump assembly 38 relative to compartment 30 containing filter means 32. As further described below, compartment 30 and pump assembly 38 are configured to be securely mounted to rear wall 16 of tank 12, and pipe fitting assembly 36 and filter means 32 are configured to be securely mounted inside interior space B of compartment 30.

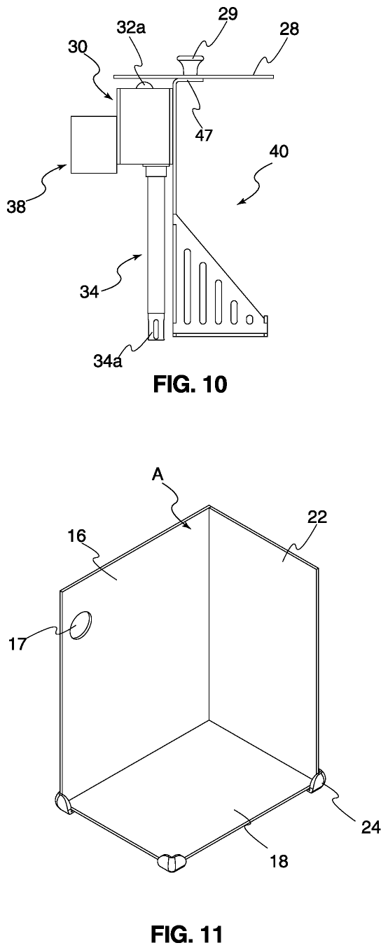

FIG. 10 is a side view illustrating a positional relationship between the assembly in FIG. 9 and rack 40 when rack 40 is disposed in interior space A of tank 12. In this figure, tank 12 is omitted to facilitate illustration of this positional relationship. This view also illustrates cover 28 with holding member 29 being attached to a top end portion 47 of rack 40 as described above. As can be appreciated from this figure, when disposed in interior space A of tank 12, track 40 is configured for placement in close proximity to compartment 30 and fluid flow pipe 34, thereby further contributing to the compact structural feature of device 10.

Pump assembly 38 may be any type of pump assembly configured for circulating the cleaning and disinfecting fluid within tank 12, including circulation of the disinfecting fluid through filter means 32 in compartment 30. For example, pump assembly 38 may be in the form of any commercially available centrifugal pump, where suction created by an impeller of the pump is a result of the centrifugal force generated by the rotation of the impeller. As the impeller rotates, it creates a decrease in pressure at an inlet of the pump, which causes fluid to be pulled into the pump. The fluid is then propelled out of the pump by the centrifugal force generated by the impeller. Pump assembly 38 can be powered electrically by a power cable connectable to an AC electrical outlet (not shown). Alternatively, pump assembly 38 can be powered by a DC battery. A pump assembly suitable for use with device 10 according to the embodiments of the present invention is preferably a motor unit and impeller assembly, such as sold under the trade name AquaClear® (https://www.amazon.com/AquaClear-Motor-Unit-Power-Filter/dp/B000069R2C?th=1). It will be appreciated, however, that are types of commercially available pump assemblies are suitable for circulating the disinfecting fluid within tank 12.

The cleaning and disinfecting fluid selected for use with device 10 is preferably one adapted for sufficiently and effectively cleaning and disinfecting the instruments and related items supported by rack 40. For example, the disinfectant sold under the trade name Barbicide® may be used as the cleaning and disinfecting fluid for use with device 10. It is understood that other commercially available cleaning and disinfecting fluids are also suitable for use with device 10.

As described above, device 10 is configured to circulate the cleaning and disinfecting fluid contained in tank 12 through filter means 32 which cleans/filters the fluid by removing particles from the fluid collected during cleaning and disinfection of instruments and related items as described herein. The fluid is circulated by action of the pump assembly 38 which causes pipe 34 to draw (e.g., suck) the fluid from interior space A of tank 12 and direct the fluid through assembly 36 and toward pump assembly 38 and filter means 32.

Filter means 32 comprises a filter case or holder configured to securely hold therein a filter element. The filter holder has a portion 32a to facilitate insertion and removal of the filter holder into and out of interior space B of compartment 30. The filter element selected is preferably one which contains a filter media adapted for filtering the specific type of cleaning and disinfecting fluid selected for use with device 10. For example, the filter element (i.e., filter pad) sold under the trade name Imagitarium® (https://www.amazon.com/s?k=imagitarium+filter+pad&hvadid=598852193512&hvdev =c&hvlocphy=1015143&hvnetw=g&hvqmt=e&hvrand=9103161892105383808&hvtargi d=kwd-381245657754&hydadcr=16169_13559671&tag=googhydr-20&ref=pd_sl_81q1wxui3s_e) may be selected for use with device 10 to filter the cleaning and disinfecting fluid. It is understood that other commercially available filters are also suitable for accomplishing this filtering process according to the present invention.

Referring to FIGS. 7, 21 and 32, compartment 30 is an enclosure formed by sidewalls 31, 33, a front wall 35, a rear wall 37 having an opening 37a, and a bottom wall 39 having an opening 39a. Compartment 30 has an open top end providing access into interior space B. Rear wall 37 is configured for integral connection to rear wall 16 of tank 12. This can be accomplished using any suitable technique, such as by bonding rear wall 37 to rear wall 16 with glue or by welding, or by using suitable fasteners. Front wall 35 is formed with an undercut portion 35a serving as an overflow region for compartment 30 when the fluid being circulated through filter element 32 fills interior space B of compartment 30. Fluid which flows over undercut portion 35a flows back into interior space A of tank 12. By this construction, undercut portion 35 provides for a more controlled flow of fluid from interior space B of compartment 30 back into interior space A of tank 12.

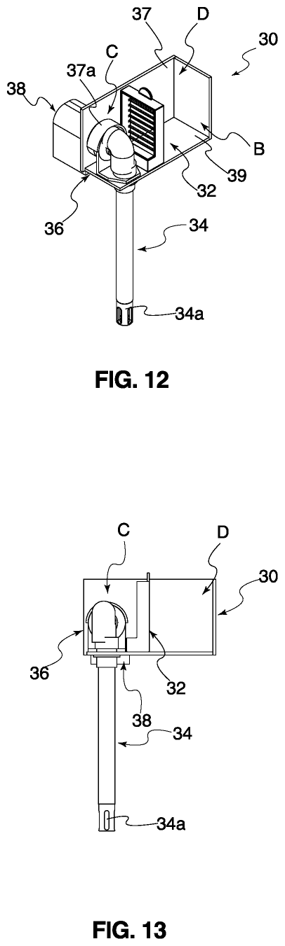

FIG. 11 is a perspective view of tank 12 with front wall 14 and left-side wall 20 omitted to provide further illustration of interior space A as well as an opening 17 formed through rear wall 16. Opening 17 is configured to be aligned with opening 37a formed in rear wall 37 of compartment 30. Pump assembly 38 has an inlet tube portion 38a with an inlet opening 38c that extends through opening 17 of tank 12 and opening 37a of compartment 30. A connecting element 38b engages inlet tube portion 38a to securely mount pump assembly 38 to rear wall 16 of tank 12 and rear wall 37 of compartment 30. The engagement between inlet tube portion 38a and connecting element 38b may be accomplished by a threaded engagement between these components and/or by suitable fasteners, for example. By this construction and corresponding positional arrangement, the inlet of pump assembly 38 securely and efficiently opens into interior space B of compartment 30 (FIGS. 12, 15, 19, 25, 28, 29, 32).

The structural details of pipe fitting assembly 36 will now be described with reference to the drawings. As shown in FIGS. 31a, 31b and 32, pipe fitting assembly 36 (hereinafter also referred to as “assembly 36”) includes a generally L-shaped base member having leg portions 36a, 36b, a generally cylindrical fitting 36c mounted to the base member, an elbow fitting 36d mounted to fitting 36c, and a generally semi-circular shaped cover member 36e connected to an upper part of leg portion 36b. Each of leg portions 36a, 36b of the base member is provided with an opening in fluid communication with respective openings 36h, 36f of fitting 36c and fitting 36d. A terminal end at the opening of fitting 36d is provided with a funnel-shaped portion 36g surrounding opening 36f. Cover member 36e partially surrounds opening 36f and portion 36g. By this construction, assembly 36 is configured to permit fluid flow into fitting 36c via opening 36h, through fitting 36d, and out of opening 36f and through portion 36g.

Referring to FIG. 32, assembly 36 is securely mounted to bottom wall 37 of compartment 30. Specifically, opening 36h of fitting 36c and the opening of leg portion 36a are aligned with one another and with opening 39a of bottom wall 39 so as to permit fluid flow therethrough. A pipe fitting 50 with an opening 52 extends through opening 39a of bottom wall 39 and into fitting 36c via opening 36h. For example, pipe fitting 50 is connected to fitting 36c via friction fit, for example. A connector 54 securely connects (e.g., tightens) fitting 50 to bottom wall 39 and fitting 36c. With pump assembly 38 being securely mounted to rear wall 16 of tank 12 and rear wall 37 of compartment 30, and with assembly 36 being securely mounted to bottom wall 37 of compartment 30, as described above, opening 36f is disposed proximate and in spaced-apart relation to opening 38c of pump assembly 38, as shown in FIG. 32 and in FIGS. 12-14, 19, 24-30. More specifically, assembly 36 is disposed proximately but is not connected to any part of pump assembly 38.

Referring again to FIG. 32, cover member 36e of assembly 36 partially surrounds both opening 38c of pump assembly 38 and opening 36f (including portion 36g) of elbow fitting 36d. Cover member 36e collaborates with funnel-shaped portion 36g to direct the flow of fluid exiting opening 36f of assembly 36 towards opening 38c of pump assembly 38 and towards filter element 32 inside interior space B of compartment 30. Cover member 36e also functions to prevent the flow of fluid exiting opening 36f of assembly 36 from spraying outwardly (i.e., vertically) toward open top end 13 of tank 12.

FIG. 12 is a perspective view of the assembly in FIG. 9, with front 35 and left-side 33 walls of compartment 30 removed to better illustrate filter element 32 and assembly 36 disposed in interior space B of compartment 30. FIG. 13 is a side view of the assembly in FIG. 12. As shown in FIGS. 12-13, when positioned inside interior space B of compartment 30, filter element 32 divides interior space B into compartment section C, which is configured to receive and hold therein the fluid to be filtered discharged from opening 36a of assembly 36 and opening 38c of pump assembly 38, and compartment section D, which is configured to receive the filtered fluid (i.e., the fluid that is passed through filter means 32). More specifically, compartment section C is disposed upstream of filter means 32 and is configured to receive the fluid to be filtered after the fluid sucked up through pipe 34 from interior space B of tank 12 by the action of pump assembly 38 is directed out of opening 36f of assembly 36 as well as fluid discharged from opening 38c of pump assembly 38. Compartment section D is disposed upstream of filter means 32 and is configured to receive the fluid after it passes through filter element 32 (i.e., the filtered fluid). The filtered fluid is then caused to flow out of compartment section D and downwardly into interior space A of tank 12 by means of undercut portion 35a (overflow regions) as described above.

By the foregoing construction and mode of operation, device 10 provides a self-contained cleaning and disinfecting system by which the disinfecting fluid is continuously circulated by means of pump assembly 38 from the region of interior space A containing the instruments (i.e., supported by rack 40) to be cleaned and disinfected, through pipe 34 and assembly 36, through filter element 32 via compartment section C by which the fluid is filtered, and into compartment section D until the fluid overflows back (via undercut portion 35a) into the region interior space A containing the instruments to repeat the fluid circulation along with the cleaning and disinfecting functions.

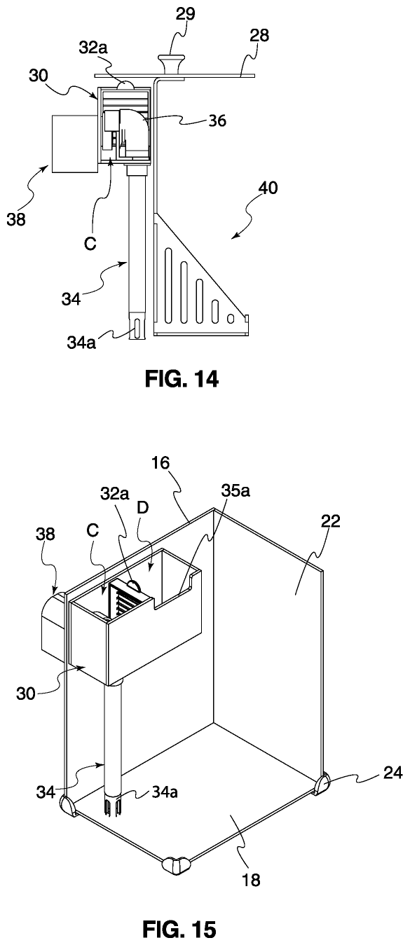

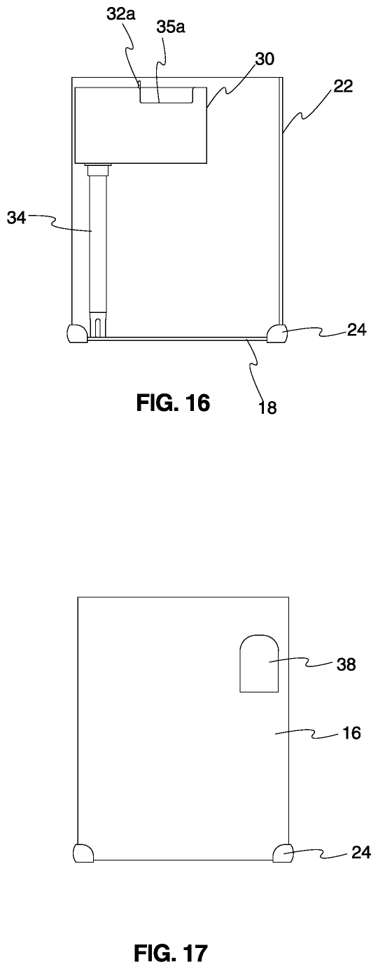

FIG. 14 is a view similar to FIG. 10 with left side wall 33 of compartment 30 removed to illustrate another view of the positional relationship between filter element 32 and assembly 36 disposed in compartment section D and pump assembly 38 mounted to compartment 30 via rear wall 16 of tank 12. FIG. 15 is a perspective view of tank 12 with front wall 14 and side wall 20 removed to provide an illustration of how pump assembly 38 and compartment 30 (containing assembly 36 and filter element 32) are mounted to rear wall 16 of tank 12. FIGS. 16-20 show front, rear, left side, top and bottom view, respectively, of the view in FIG. 15. FIGS. 14-20 further illustrate the compact construction of device 10 according to the embodiment of the present invention.

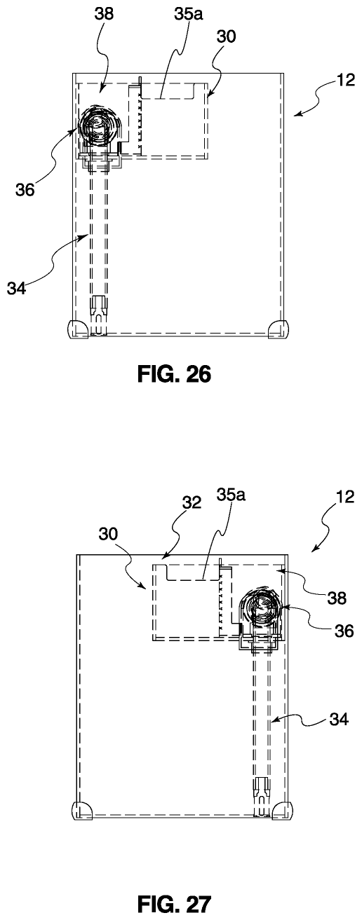

FIGS. 21-30 show device 100 according to the second embodiment of the present invention. As noted above, device 100 has the same components as device 10 of the first embodiment described above with reference to FIGS. 1-20, where the same reference numbers represent the same elements throughout. The only difference between the first and second embodiments is that in device 100 the walls of tank 12, cover 28 and compartment 30 are made of a transparent (see-through) material, whereas in device 10 tank 12, cover 28 and compartment 30 are not made of a transparent material. FIGS. 21-24 are views similar to the views in FIGS. 7-10, respectively. FIG. 25 is a perspective view similar to FIG. 15, except that in FIG. 15 front wall 14 and left side wall 20 of tank 14 have been omitted. FIGS. 26-30 are front, rear, left side, top and bottom view of the illustration in FIG. 25. The various features and functions of the components of device 100 are as described above for the corresponding components of device 10.

As described above, FIGS. 31a, 31b and 32 show details of compartment 30, assembly 36, and pump assembly 38 used in devices 10 and 100 according to the embodiments of the present invention. The types of cleaning and disinfecting fluid, filter element and pump assembly described above for use with device 10 may also be selected for use with device 100.

Suitable, durable plastic materials may be used to construct the various components described above for devices 10 and 100. For example, pipe 34 and assembly 36 may be made of polyvinyl chloride (PVC). Tank 12, cover 28, compartment 30, filter holder for filter means 32, and rack 40 can be made of an acrylic material (e.g., Plexiglass®). It is understood that other commercially available materials are suitable for the components of devices 10 and 100.

It is also understood that the shapes and configurations of the components for devices 10 and 100 can be varied from those illustrated in the figures. For example, while the embodiments illustrated in the figures show tank 12 having a generally rectangular configuration, tank 12 can be configured with a cylindrical or other suitable configuration. Cover 28 and rack 40 are also adapted to be configured so as to conform to the specific configuration selected for tank 12.

It is further understood that devices 10 and 100 are not limited to any specific dimensions. For example, upon selection of the dimensions for tank 12, suitable dimensions are selected for the remaining components of devices 10 and 100 to permit accommodation of the components to and inside tank 12. As a specific example, rack 40 can be configured with a single compartment instead of two compartments 41, 43.

An example of the use and operation of devices 10 and 100 according to the embodiments of the present invention is described below. With components 30, 32, 34, 36, 38 of devices 10 and 100 assembled relative to tank 12, rack 40 is removed (i.e., lifted) from inner space A of tank 12 by lifting cover 28 via holding member 29. Space A of tank 12 is filled with the cleaning and disinfecting fluid (e.g., Barbicide®) until the fluid completely covers at least opening 36f of assembly 36 and opening 38c of pump assembly 38 (i.e., openings 36f and 38c are completely submerged in the fluid). After placing the desired instruments to be cleaned and disenficted on compartments 41, 43 of rack 40, rack 40 is inserted into space A of tank 12 by means of holding member 29 until cover 28 covers top end 13 of tank 12, as shown in FIGS. 1-6. At this point, the instruments are submerged within the fluid in tank 12 and undergo cleaning and disinfection by the fluid, causing any debris associated with the instruments to be released from the instruments and flow into tank 12. Pump assembly 38 is powered (AC or DC power) to begin circulation of the cleaning and disinfecting fluid within tank 12.

During circulation, pump assembly 38 causes the fluid containing any debris from the instruments to be drawn from space A of tank 12 by pipe 34 via inlet 34a and directed toward assembly 36 and through opening 36f of assembly 36. The fluid exiting opening 36f is caused to flow toward opening 38c of pump assembly 38 and toward filter means 32 by the action of funnel-shaped portion 36g surrounding opening 36f and the impeller of pump assembly 38. Specifically, the impeller creates suction as a result of the centrifugal force generated by the rotation of the impeller. As the impeller rotates, it creates a decrease in pressure at opening 38c of pump assembly 38, which causes the fluid to be pulled into the pump assembly. The fluid is then propelled out of opening 38c of pump assembly 38 by the centrifugal force generated by the impeller. The continuous flow of the fluid out of opening 36f of assembly 36 and out of opening 38c of pump assembly 38 is consequently forced into and begins to fill compartment section C while being directed toward and pushed through filter means 32. The fluid filling compartment section C continuously passes through filter 32 where the debris from the instruments contained in the fluid is collected by the filter media in filter means 32, resulting in the fluid being filtered and then flowing into compartment section D. The debris may be foreign substances such as loose hair and the like which ordinarily accumulates in and on most barber instruments, for example, to be cleaned and disinfected.

As it fills compartment section D, the filtered fluid is then caused to flow out of compartment section D in a controlled manner via undercut portion 35a of compartment 30 and downward toward the instruments supported by rack 40. By this process, the debris from the instruments is filtered by filter means 32 from the fluid before the filtered fluid is returned down into interior space A of tank 12 to again perform its cleaning and disinfecting function in a continuous matter within the self-contained environment of devices 10 and 100. Filter means 32, which is of a type selected so as to be able to efficiently filter any debris from the fluid passing therethrough, can be easily removed from compartment 32 periodically as necessary for cleaning and/or replacement.

According to a feature of the present invention, undercut portion 35a (e.g., FIGS. 7, 9, 15, 21, 23, 25, 27) defines an overflow region of compartment 30 that allows for the controlled overflow of the filtered fluid from interior space B of compartment 30 down towards the instruments supported by rack 40. Specifically, the operation of the means for circulating the disinfecting fluid (e.g., pipe 34, assembly 36, pump assembly 38) along with its positional relationship relative to compartment 30 and filter means 32 in interior space A of tank 12 enables for the controlled flow of the filtered fluid only from the overflow region 35a of compartment 30. Such a controlled flow feature prevents the unfiltered fluid from overflowing from compartment C before it is continuously forced through filter means 32 to undergo the filtering process. In addition to providing for the control flow of the filtered fluid from compartment 30 into space A of tank 12, the flow of the filtered fluid via the overflow region 35a generates an aesthetically pleasing and decorative cascade overflow effect.

As another feature of the present invention, interior space A of tank 12 can be provided with a lighting system to generate an aesthetically pleasing lighting effect as the fluid circulates within the tank, including when the filtered fluid overflows from undercut portion 30a and down toward the instruments supported by rack 40, thereby further enhancing the cascade overflow effect described above.

It will be appreciated that the components of devices 10 and 100 described herein with reference to the drawings can be produced in various sizes and shapes and with different materials incorporating different application considerations without departing from the objectives of the present invention. In this regard, the selection of the dimensions, configuration, and materials selected for cover 28, compartment section 30, filter means 32, pipe 34, assembly 36, pump assembly 38, and rack 40, for example, will depend on the dimensions, configurations, and materials selected for tank 12.

It is appreciated from the foregoing description with reference to the drawings that the present invention provides cleaning and disinfecting devices especially adapted for use with but not limited to barber instruments, in which the instruments may be placed and continuously and automatically cleaned and disinfected in a minimum amount of time and more economically as compared to the conventional art.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A cleaning and disinfecting device comprising:
a tank having an open top end providing access into a first interior space configured to receive therein a fluid;
instrument support means configured to be removably inserted into the first interior space of the tank for supporting at least one instrument to be cleaned and disinfected by the fluid;.
a compartment disposed within the first interior space of the tank, the compartment having a second interior space;
filter means removably disposed in the second interior space of the compartment; and
means for circulating the fluid within the first interior space of the tank through the filter means to filter the fluid by removing therefrom debris collected by the fluid when cleaning and disinfecting the instrument.

2. The device of claim 1, further comprising means for removably inserting and lifting the instrument support means into and out of the first interior space of the tank.

3. The device of claim 1, wherein instrument support means comprises a rack having at least one compartment for supporting the instrument.

4. The device of claim 3, wherein the means for removably inserting and lifting the instrument support means a cover integrally connected to the rack for covering the open top end of the tank.

5. The device of claim 1, wherein the means for circulating the fluid comprises a flow pipe having an inlet opening, a pipe fitting assembly having an outlet opening in fluid communication with the flow pipe, and a pump assembly for pumping the fluid disposed in the first interior space of the tank through the flow pipe via the inlet opening and out of the outlet opening of the pipe fitting assembly.

6. The device of claim 1, wherein the filter means comprises a filter element that separates the second interior space into a first compartment section and a second compartment section; wherein the pipe fitting assembly is disposed in the first compartment section and the pump assembly has an intake and discharge port opening into the first compartment section; and wherein the pumped fluid flows into the first compartment section and through the filter means to filter the fluid.

7. The device of claim 6, wherein the compartment is disposed proximate the open top end of the tank and has a wall provided with an undercut portion defining an overflow region of the compartment via which the filtered fluid is allowed to flow in a controlled manner downwardly back into the first interior space of the tank.

8. The device of claim 1, wherein the fluid comprises a cleaning and disinfecting fluid.

9. The device of claim 8, wherein the cleaning and disinfecting fluid is Barbicide®.

10. The device of claim 1, wherein the fluid is a cleaning and sterilizing fluid.

11. The device of claim 1, wherein the at least one instrument comprises an instrument used in the beauty industry.

12. The device of claim 1, wherein the at least one instrument is a barber instrument.

13. The device of claim 1, wherein the at least one instrument is a nail care instrument.

14. The device of claim 1, wherein each of the tank and the compartment comprises walls made of a transparent material.

15. The device of claim 1, wherein the filter means separates the compartment into a first compartment section and a second compartment section; wherein the means for circulating the fluid comprises a pipe fitting assembly mounted in the first compartment section and having a fluid outlet opening, and a pump assembly mounted to a wall of the tank and having an intake and discharge port opening into the first compartment section and disposed proximate and in spaced-apart relation to the fluid outlet opening of the pipe fitting assembly.

16. A cleaning and disinfecting device comprising:
a tank having an open top end providing access into a first interior space configured to receive therein a fluid;
a rack configured to be removably inserted into the first interior space of the tank for supporting at least one instrument to be cleaned and disinfected by the fluid;.
a compartment disposed within the first interior space of the tank, the compartment having a second interior space;
a filter removably disposed in the second interior space of the compartment;
a flow pipe having an inlet opening;
a pipe fitting assembly having an outlet opening in fluid communication with the flow pipe; and
a pump assembly for pumping the fluid disposed in the first interior space of the tank through the flow pipe via the inlet opening and out of the outlet opening of the pipe fitting assembly.

17. The cleaning and disinfecting device of claim 16, wherein the filter comprises a filter element that separates the second interior space into a first compartment section and a second compartment section; wherein the pipe fitting assembly is disposed in the first compartment section and the pump assembly has an intake and discharge port opening into the first compartment section; and wherein the pumped fluid flows into the first compartment section and through the filter element to filter the fluid.

18. The device of claim 16, wherein the fluid comprises a cleaning and disinfecting fluid.

19. The device of claim 18, wherein the cleaning and disinfecting fluid is Barbicide®.

20. The device of claim 16, wherein the fluid is a cleaning and sterilizing fluid; and
wherein the at least one instrument comprises an instrument used in the beauty industry.
